(19) United States
(12) Patent Application Publication
(10) Pub. No.: US 2019/0284125 A1
(43) Pub. Date: Sep. 19, 2019

(54) Amine Derivatives of The Beta-Farnesene

(71) Applicant: Clariant International Ltd., Muttenz (CH)

(72) Inventors: Dirk LEINWEBER, Kelkheim (DE); Steffen ROMANSKI, Wesel (DE); Andreas VORHOLT, Dortmund (DE); Arno BEHR, Dortmund (DE); Thiemo Alexander FASSBACH, Mülheim an der Ruhr (DE)

(73) Assignee: Clariant International Ltd., Muttenz (CH)

(21) Appl. No.: 16/465,563

(22) PCT Filed: Dec. 1, 2017
(86) PCT No.: PCT/EP2017/081218

§ 371 Date: May 30, 2019

(30) Foreign Application Priority Data
Dec. 5, 2016 (DE) 10 2016 224 089.7

(51) Int. Cl. C07C 209/60; B01J 31/22; B01J 27/10; B01J 31/24

(52) U.S. Cl. CPC C07C 209/60; B01J 31/2239; B01J 27/10; C07C 211/21; B01J 2531/847; B01J 2531/824; B01J 2231/44; B01J 31/2404

(57) ABSTRACT

The invention provides a process for preparing farnesylamines by reacting β-farnesene with one or more amines in the presence of a transition metal catalyst from transition group 10 at a temperature in the range from 60 to 150° C.

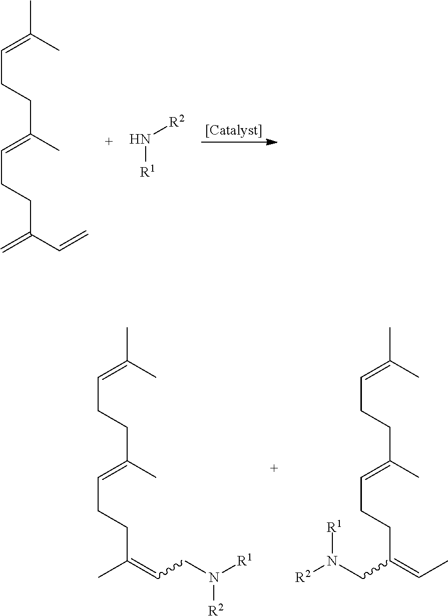

The present invention relates to the preparation of amine derivatives of β-farnesene by metal-catalyzed addition of amines on to the 1,3-diene unit of the farnesene. These products are, for instance, important intermediates in fragrance synthesis, and downstream products of the farnesylamines also have surfactant properties.

Industrially, terpenylamines are prepared by reaction of terpenes, such as β-myrcene, and an amine in the presence of elemental sodium. In this way, in the course of the Takasago process, (−)-menthol is prepared starting from β-myrcene in a multistage procedure.

In analogy to this, the hydroamination of β-farnesene with organolithium compounds is also described in various publications (EP-A-2 894 143). The products of the examples stated are intermediates in fragrance synthesis.

One of the disadvantages of base-catalyzed hydroaminations is the incompatibility with protic solvents, such as alcohols. Here, the corresponding alkoxides are formed before the amine can be deprotonated. Furthermore, the use of bases also has consequences for the spectrum of substrates that can be used. Amines functionalized with hydroxyl or carboxyl groups (diethanolamine or sarcosine, for example) would undergo preferential deprotonation at these groups before base-catalyzed hydroamination was possible.

Furthermore, the use of elemental sodium or butyl lithium imposes exacting demands on the inertness of the reaction vessel and of the materials used, so as to minimize explosion risks, and also on the construction materials used, which must be resistant to such reducing agents.

Transition-metal-catalyzed processes for hydroamination tend generally to belong to the realm of academic research. Among the systems to have emerged as active systems for 1,3-dienes are nickel and palladium catalysts with monophosphine and diphosphine ligands (J. Pawlas, Y. Nakao, M. Kawatsura, J. F. Hartwig, J. Am. Chem. Soc. 2002, 124, 3669-3679).

Of industrial significance in particular are processes which allow conversion of a wide spectrum of amines, such as, for example, alkylamines, alkanolamines, especially sugar amines, and amino acids. Reactions which require particular functionalities on the nitrogen, such as anilines or hydrazines, for example, are not useful, owing to the high toxicity of the reactants employed.

Other transition-metal-catalyzed hydroaminations utilize rhodium catalysts, but the latter can often be used only for intramolecular hydroaminations (US 2012/0190854). There are also descriptions of ruthenium catalysts, but in that case high concentrations of catalyst (>5 mol %) are required, and they must also be activated by means of cocatalysts (WO 2005/077885). A key disadvantage of homogeneously catalyzed processes is that it is often not possible to separate off and reuse the homogeneous transition metal catalyst.

Industrially, hydroaminations are often conducted over heterogeneous catalysts, but the activity of these catalysts suffers from a low rate of diffusion to the active centers, meaning that the physical properties of the heterogeneous catalyst must be tailored precisely to the substrates and products.

In 2010, Behr, Johnen and Rentmeister presented a hydroamination of a terpene using homogeneous transition metal catalysts. Specifically, they hydroaminated β-myrcene over a palladium/diphosphine catalyst with morpholine (A. Behr, L. Johnen, N. Rentmeister, Adv. Synth. Catal. 2010, 352, 2062-2072).

One substrate of particular interest for transition-metal-catalyzed hydroamination is farnesene, which can be obtained industrially by a fermentation process from renewable raw materials. The use of β-farnesene in hydroamination is a particular challenge, since the reaction of the highly apolar farnesene with polar amines can lead to inhomogeneous reaction solutions in which efficient conversions are impossible.

It is an object of the present invention, therefore, to provide an economic, transition-metal-catalyzed process for the hydroamination of β-farnesene that can be carried out with good yields even in protic solvents and using functionalized amines.

Surprisingly it has been found that transition-metal-catalyzed hydroaminations of β-farnesene are possible, despite the chain length for this substrate resulting in very large polarity differences between the terpene and the amines employed.

The present invention therefore provides a process for preparing farnesylamines by reacting β-farnesene with one or more amines in the presence of a transition metal catalyst from transition group 10 at a temperature in the range from 60 to 150° C.

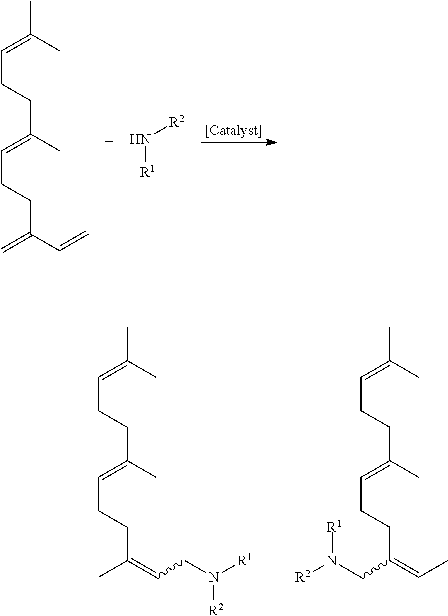

A broad range of amines come into consideration as the nucleophile, and may contain substituents with a wide variety of different chain lengths and functionalizations. For example, alkylamines with short chains (dimethylamine, diethylamine), amino sugars (N-methylglucamine) or amino acids (sarcosine) may be added on to the 1,3-diene unit of the β-farnesene. The resultant farnesylamines can be used as surfactants based on renewable raw materials, or can be converted by further reactions into surfactants based on renewable raw materials.

For the hydroamination it is possible to use all amines which possess a proton on the nitrogen and can therefore be added on to a double bond.

In the amine of the formula HNR¹R², R¹ and R² independently of one another are H or branched, cyclic, heterocyclic, linear, optionally substituted alkyl radicals which are in saturated or unsaturated form and have a chain length in the range of C₁-C₃₀ or independently of one another are an aromatic or heteroaromatic radical, or one of the radicals R¹ and R² is NR²R³, in which R² and R³ independently of one another are H or branched, cyclic, linear, optionally substituted alkyl radicals or aromatic or heteroaromatic radicals which are in saturated or unsaturated form and have a chain length in the range of C₁-C₃₀, preferably in the range of C₁-C₁₅, more particularly in the range of C₁-C₁₀.

These alkyl radicals may be unfunctionalized or may carry functional groups, such as, for example, alcohols, ester, acid or keto groups, or aromatic substituents. The radicals R¹ and R² may also be aromatic substituents, such as phenyl or benzyl radicals or heteroaryl radicals, such as pyrrole, for example, and these aromatics may carry functional groups, such as alcohols, ester, acid or keto groups.

Furthermore, the amine component may also comprise diamines, such as ethylenediamine, or secondary amines, such as piperazine, preference being given to unbranched monoamines, such as diethylamine, dimethylamine, dipropylamine, and dibutylamine, for example.

The hydroamination can be carried out in various solvents, such as in organic aliphatic or aromatic hydrocarbons, more particularly in polar or polar-aprotic hydrocarbons, or ionic liquids. Preferred solvents are for example as follows:
aliphatic hydrocarbons, such as n-pentane, n-hexane, n-octane, isooctane;
alcohols, such as methanol, ethanol, isopropanol, tert-butanol;
aromatic hydrocarbons, such as toluene, xylenes, mesitylene;
nitriles, such as acetonitrile, 3-methoxypropionitrile;
amides, such as dimethylformamide, dimethylacetamide, and so on;
ether compounds, such as diethyl ether, methyl tert-butyl ether, anisole;
carbonates, such as ethylene, propylene or butylene carbonate;
ionic liquids, such as dimethyl ammonium carbamate (Dimcarb)
strongly polar solvents, such as dimethyl sulfoxide, N-methylpyrrolidone.

Especially preferred are anisole, dimethylformamide, dimethylacetamide, methanol, isopropanol, dioxane, dimethyl sulfoxide (DMSO), dimethyl ammonium carbamate (Dimcarb), and acetonitrile.

Depending on the amine used, the reaction may even be carried out entirely without solvent, as is the case, for example, when using Dimcarb, since in that case the Dimcarb is employed both as solvent and as substrate.

In one preferred embodiment, the solvent is used in a one- to ten-fold excess relative to the starting products, more particularly in a two- to five-fold excess.

Employed as catalyst are transition metals from transition group 10 of the Periodic Table, more particularly nickel, palladium or platinum precursors.

The transition metal catalyst is completely in solution in the reaction mixture and is modified by an organic ligand.

In accordance with the invention, the catalyst and the farnesene are used in a molar ratio of 1:10 to 1:1000, more particularly 1:200, very preferably 1:125.

Preferred precursors are selected from the following group:
nickel precursors, for example Ni⁰(cod)₂, Ni^{II}(acac)₂, Ni^{II}(hfacac)₂, Ni^{II}Cl₂;
palladium precursors, for example Pd⁰₂dba₃, Pd^{II}(acac)₂, Pd^{II}(hfacac)₂, Pd^{II}(tfa)₂, Pd^{II}Cl₂;
platinum precursors, for example Pt^{II}Cl₂, Pt^{II}(cod)Cl₂, Pt^{II}(acac)₂, K₂Pt^{II}Cl₄; to name but a few.

Particular preference is given to palladium precursors having fluorinated leaving groups, such as Pd^{II}(tfa)₂ or Pd^{II}(hfacac)₂.

In one preferred embodiment it is possible to recycle the catalyst used, after the reaction.

One way of removing the catalyst is to use a polar phase and a catalyst having polar ligands for the synthesis of apolar products. The apolar products can then be extracted with apolar hydrocarbons such as n-decane or n-dodecane, for example. In the extraction, the catalyst is retained in the polar phase and can be used for further reactions.

Hence when using ammonium carbamates, more particularly dimethyl ammonium carbamate, for example, and correspondingly sulfonated ligands, such as TPPMS, TPPTS or DPPBTS, for example, it is possible to immobilize the catalyst complex in the polar phase, to separate it from the product, which forms its own phase, which may then be extracted with additional Dimcarb, and to use it again in a further reaction. In that case, fresh farnesene is added to the polar phase and a new reaction run is initiated.

Another way of removing the homogeneous catalyst is to use TMS (=thermomorphic multicomponent solvent) systems. In these systems, two solvents of different polarity are used (for example acetonitrile/n-heptane or DMF/n-decane), which are as one phase at reaction temperature and as two phases at separation temperature. Accordingly, the products and the hydroamination catalyst can be accumulated in different phases (T. Farber, O. Riechert, T. Zeiner, G. Sadowski, A. Behr, A. J. Vorholt, Chem. Eng. Res. Des. 2016, 112, 263-273.).

The transition-metal-catalyzed hydroamination of the invention can be carried out either with or without ligands. Preference is given to using phosphorus ligands. The following table contains a number of selected examples, along with their structures:

| Ligand | Structure | IUPAC name |
|---|---|---|
| PPh₃ | | triphenylphosphine |
| TPPMS | 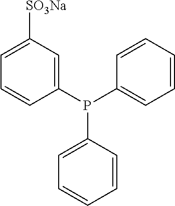 | sodium 3- (diphenylphosphanyl)benzenesulfonate |
| TPPTS | 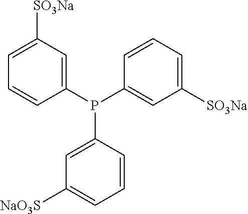 | sodium 3,3′,3″- phosphanetribenzyltrisulfonate |
| P(OPh)₃ | 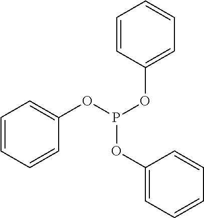 [SMILES: c1ccc(OP(Oc2ccccc2)Oc2ccccc2)cc1] | triphenylphosphite |
| TOMPP | 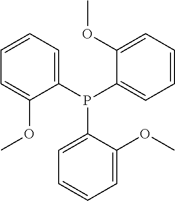 [SMILES: COc1ccccc1P(c1ccccc1OC)c1ccccc1OC] | tris(ortho-methoxyphenyl)phosphine |
| PCy₃ | 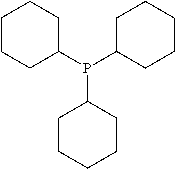 [SMILES: C1CCC(P(C2CCCCC2)C2CCCCC2)CC1] | tricyclohexylphosphine |
| PEt₃ | 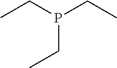 | triethylphosphine |
| PtBu₃ | 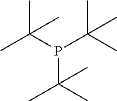 | tri-tert-butylphosphine |
| DPPE | 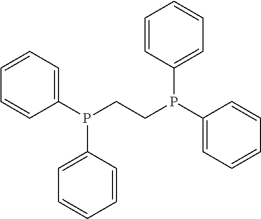 [SMILES: c1ccc(P(CCP(c2ccccc2)c2ccccc2)c2ccccc2)cc1] | bis(diphenylphosphino)ethane |
| DPPP | 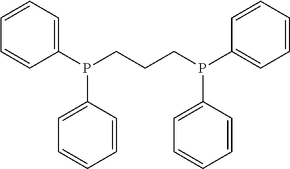 [SMILES: c1ccc(P(CCCP(c2ccccc2)c2ccccc2)c2ccccc2)cc1] | bis(diphenylphosphino)propane |
| DPPB | 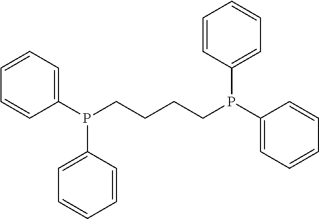 [SMILES: c1ccc(P(CCCCP(c2ccccc2)c2ccccc2)c2ccccc2)cc1] | bis(diphenylphosphino)butane |
| DPPBTS | 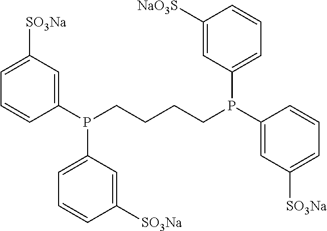 | bis(diphenylphosphino)butane tetrasulfonate |
| DPPH | 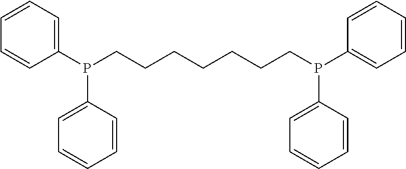 [SMILES: c1ccc(P(CCCCCCCP(c2ccccc2)c2ccccc2)c2ccccc2)cc1] | bis(diphenylphosphino)heptane |
| DPPF | 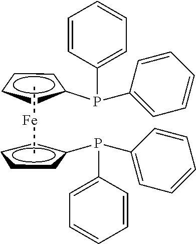 | 1,1′- bis(diphenylphosphino)ferrocene |
| Xantphos | 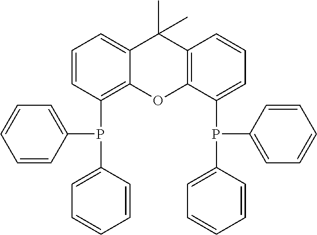 [SMILES: CC1(C)c2cccc(P(c3ccccc3)c3ccccc3)c2Oc2c(P(c3ccccc3)c3ccccc3)cccc21] | (9,9-dimethyl-9H-xanthene-4,5- diyl)bis(diphenylphosphine) |
| Sulfo- Xantphos | 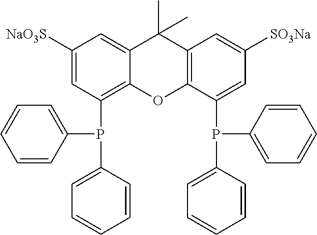 | sodium 4,5- bis(diphenylphosphanyl)-9,9- dimethyl-9H-xanthene-2,7- disulfonate |
| NiXantphos | 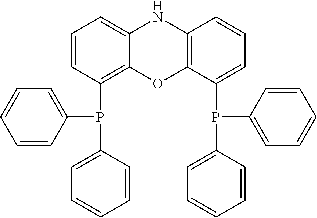 [SMILES: c1ccc(P(c2ccccc2)c2cccc3c2Oc2c(cccc2P(c2ccccc2)c2ccccc2)N3)cc1] | 4,6-bis(diphenylphosphanyl)-10H- phenoxazine |
| DPEphos | 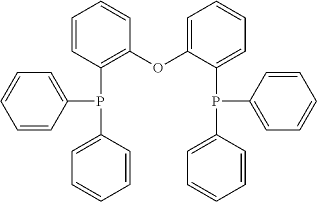 [SMILES: c1ccc(P(c2ccccc2)c2ccccc2Oc2ccccc2P(c2ccccc2)c2ccccc2)cc1] | (oxybis(2,1- phenyl))bis(diphenylphosphane) |

In one preferred embodiment the diphosphines have a bite angle of close to 100°, such as DPEphos or DPPB, for example.

The reaction of hydroamination over transition metal catalysts is determined by the amount-of-substance ratios of the reactants involved. A critical part here is played both by the substrate ratios and by the metal/ligand ratio.

Customary ligand-to-metal ratios are in the range from 1:1 to 1:50 (palladium/ligand [mol/mol]), more particularly in the 1:2 to 1:30 range, especially preferably in the range from 1:2 to 1:16 mol/mol.

Customary amounts of substance of β-farnesene:amine used are in the range from 1:1 to 1:10 mol/mol, more particularly in the range from 1:2 to 1:5 mol/mol.

The amines in accordance with the invention are used in the same amount-of-substance concentration or in a slight excess. In one preferred embodiment, an amine excess with a factor of 2 to 5 has beneficial consequences for the conversion of the β-farnesene.

The reaction is conducted preferably in the presence of an inert gas, more particularly argon or nitrogen.

The hydroamination may take place under a pressure in the range from 1 to 10 bar, more particularly at 2 to 8 bar, very preferably at 3-6 bar.

The reaction time is generally 1 to 16 hours, preferably 2 to 10, more particularly 3 to 8 hours.

The hydroamination of β-farnesene in accordance with the invention is customarily carried out at a temperature in the range from 50 to 150° C., preferably at 60 to 120° C., more particularly at 70 to 100° C.

The resulting amine derivatives of β-farnesene may optionally be modified further and used as surface-active substances in numerous fields of application.

For instance they may be optionally further modified and used as surface-active substances in numerous fields of application, such as, for example, personal care, especially hair care and skin care applications, automatic dishwashing, hard surface cleaning, or in the area of oil field chemicals.

The working examples which follow serve merely to illustrate the invention and are not intended to confine it to the content of the examples.

### EXAMPLES

Key to abbreviations used:

| Abbreviation | Meaning |
|---|---|
| acac | acetylacetonate |
| cod | 1,5-cyclooctadiene |
| dba | dibenzylideneacetone |
| DMF | N,N-dimethylformamide |
| g | gram |
| h | hour |
| hfacac | 1,1,1,5,5,5,-hexafluoroacetylacetonate |
| mg | milligram |
| mL | milliliter |
| mmol | millimol |
| mol % | mole percent |
| rpm | revolutions per minute |
| tfa | trifluoroacetate |
| Dimcarb | dimethylammonium dimethylcarbamate |

In a typical experiment in accordance with the invention, the reaction mixture is weighed into a 25 mL steel autoclave with magnetic stirring kernel. The precursor is introduced together with the ligand and is dissolved in the solvent. Then the β-farnesene and the amine are added. The reactor is closed and subjected where appropriate to argon pressure. The reactor is heated to reaction temperature and stirred with a heating stirrer. The reaction is ended by cooling the reactor to room temperature and degassing it. The reaction mixture present may be analyzed by gas chromatography.

Following removal of the solvent under a high vacuum, the product mixture can be purified by column chromatography.

### Example 1

In a 25 mL steel autoclave, 16.6 mg of Pd(hfacac)₂ and 109.3 mg of DPPB are weighed out and dissolved in 5 mL of DMF. Then 817.4 mg of β-farnesene and 296.9 mg of diethylamine are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 863.2 mg (78%) of the hydroamination products.

### Example 2

In a 25 mL steel autoclave, 10.6 mg of Pd(tfa)₂ and 137.8 mg of DPEphos are weighed out and dissolved in 5 mL of methanol. Then 817.7 mg of β-farnesene and 297.1 mg of diethylamine are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 960.4 mg (89%) of the hydroamination products.

### Example 3

In a 25 mL steel autoclave, 10.5 mg of Pd(tfa)₂ and 137.7 mg of DPEphos are weighed out and dissolved in 5 mL of anisole. Then 816.8 mg of β-farnesene and 516.6 mg of dibutylamine are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 960.8 mg (72%) of the hydroamination products.

### Example 4

In a 25 mL steel autoclave, 10.7 mg of Pd(tfa)₂ and 137.5 mg of DPEphos are weighed out and dissolved in 5 mL of DMF. Then 817.2 mg of β-farnesene and 965.3 mg of dioctylamine are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 998.6 mg (56%) of the hydroamination products.

### Example 5

In a 25 mL steel autoclave, 11 mg of Pd(tfa)₂ and 138.2 mg of DPEphos are weighed out and dissolved in 5 mL of acetonitrile. Then 818.2 mg of β-farnesene and 388.9 mg of diallylamine are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 410 mg (34%) of the hydroamination products.

### Example 6

In a 25 mL steel autoclave, 11 mg of Pd(tfa)₂ and 137.9 mg of DPEphos are weighed out and dissolved in 5 mL of DMF. Then 818.0 mg of β-farnesene and 420.8 mg of diethanolamine are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 643 mg (52%) of the hydroamination products.

### Example 7

In a 25 mL steel autoclave, 11 mg of Pd(tfa)₂ and 138.0 mg of DPEphos are weighed out and dissolved in 5 mL of acetonitrile. Then 817.5 mg of β-farnesene and 536.9 mg of dimethylammonium dimethylcarbamate are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 978 mg (98%) of the hydroamination products.

### Example 8

In a 25 mL steel autoclave, 11.0 mg of Pd(tfa)₂ and 137.9 mg of DPEphos are weighed out and dissolved in 5 mL of anisole. Then 818.2 mg of β-farnesene and 845.0 mg of N-methylglucamine are added. The reactor is closed and subjected to an argon pressure of 5 bar.

The reactor is heated to 100° C. for 16 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 351 mg (22%) of the hydroamination products.

### Example 9

In a 25 mL steel autoclave, 11.1 mg of Pd(tfa)₂ and 138.2 mg of DPEphos are weighed out without solvent. Then 1636.2 mg of β-farnesene and 601.5 mg of diethylamine are added. The reactor is closed and subjected to an argon pressure of 5 bar.

The reactor is heated to 100° C. for 4 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 1258 mg (65%) of the hydroamination products.

### Example 10

In a 25 mL steel autoclave, 10.6 mg of Pd(tfa)₂ and 137.7 mg of DPPB are weighed out without solvent. Then 1602.4 mg of β-farnesene and 2016.2 mg of dimethylammonium dimethylcarbamate are added. The reactor is closed and subjected to an argon pressure of 5 bar.

The reactor is heated to 100° C. for 4 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 1621 mg (80%) of the hydroamination products.

### Example 11

In a 25 mL steel autoclave, 11.3 mg of Pd(tfa)₂ and 137.7 mg of DPEphos are weighed out and dissolved in 5 mL of DMF. Then 818.8 mg of farnesene and 857.6 mg of benzylamine are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 199 mg (16%) of the monoalkylated product and 495 mg (48%) of the dialkylated product.

### Example 12

In a 25 mL steel autoclave, 10.6 mg of Pd(tfa)₂ and 100.0 mg of DPPBTS and subsequently 3066.4 mg of farnesene and 6039.2 mg of Dimcarb (=dimethylammonium dimethylcarbamate) are added. The reactor is closed and heated to 100° C. for 3 h, stirred at 500 rpm by magnetic stirrer. To end the reaction, the reactor is cooled to room temperature and the gas pressure which has arisen is discharged.

The reaction mixture is transferred under an argon countercurrent to a Schlenk vessel, where spontaneous phase separation takes place. The lower, polar phase is returned to the reactor. The apolar product phase is extracted with 1073.8 mg of Dimcarb. After further phase separation, the polar phase is transferred together with 3065.7 mg of farnesene into the reactor, and a new reaction run is commenced.

The product phase can be purified by column chromatography. Over a number of runs, between 2543 and 3366 mg of products are obtained.

### Example 13

In a 25 mL steel autoclave, 12.6 mg of Pd(tfa)₂ and 137.8 mg of DPEphos are weighed out and dissolved in 5 mL of DMF. Then 818.0 mg of farnesene and 745.0 mg of aniline are added. The reactor is closed and subjected to an argon pressure of 5 bar.

The reactor is heated to 100° C. for 5 h with stirring at 500 rpm by magnetic stirrer. To end the reaction, the reactor is cooled to room temperature and then the argon is cautiously discharged.

The reaction solution obtained is freed from the solvent under reduced pressure and the product is purified by column chromatography. This gives 916.3 mg (77%) of the monoalkylated products. The dialkylated product was not observed.

1. A process for preparing a farnesylamine by reacting β-famesene with at least one amine in the presence of a transition metal catalyst from transition group 10 at a temperature in the range from 60 to 150° C.

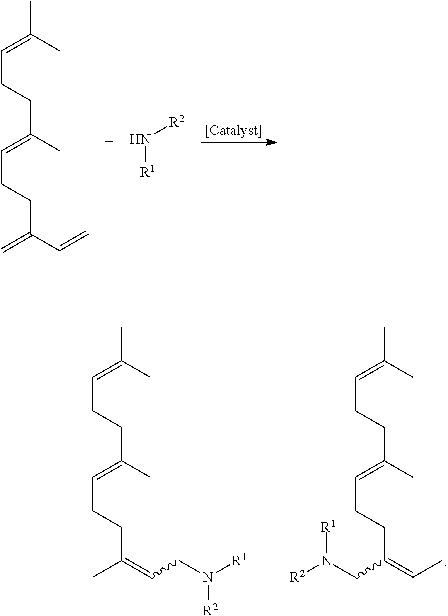

2. The process as claimed in claim 1, wherein the transition metal catalyst is selected from the group consisting of nickel, palladium and platinum precursors.

3. The process as claimed in claim 1, wherein the transition metal catalyst is completely dissolved in the reaction mixture and is modified by an organic ligand.

4. The process as claimed in claim 1, wherein the precursors are selected from the group consisting of Ni⁰(cod)₂, Ni^{II}(acac)₂, Ni^{II}(hfacac)₂, Ni^{II}Cl₂, Pd⁰₂dba₃, Pd^{II}(acac)₂, Pd^{II}(hfacac)₂, Pd^{II}(tfa)₂, Pd^{II}Cl₂, Pt^{II}Cl₂, Pt^{II}(cod)Cl₂, Pt^{II}(acac)₂, and K₂Pt^{II}Cl₄.

5. The process as claimed in claim 1, wherein the reacting is carried out in at least one solvent.

6. The process as claimed in claim 4, wherein at least one solvent is selected the group consisting aliphatic or aromatic, polar or polar-aprotic hydrocarbons, and ionic liquids.

7. The process as claimed in claim 1, wherein an ammonium carbamate and no further solvent is used for the reacting.

8. The process as claimed in claim 1, wherein an amine of the formula HNR¹R² is used, in which R¹ and R² independently of one another are H or branched, cyclic, heterocyclic, linear, optionally substituted alkyl radicals which are in saturated or unsaturated form and have a chain length in the range of C₁-C₃₀ or independently of one another are an aromatic or heteroaromatic radical, or one of the radicals R¹ and R² is NR²R³, in which R² and R³ independently of one another are H or branched, cyclic, linear, optionally substituted alkyl radicals or aromatic or heteroaromatic radicals which are in saturated or unsaturated form and have a chain length in the range of C₁-C₃₀.

9. The process as claimed in claim 1, wherein the process is carried out in the presence of a phosphorus ligand.

10. The process as claimed in claim 9, wherein the phosphorus ligand is selected from the group consisting of triphenylphosphine, triphenyl phosphite, tris(ortho-methoxyphenyl)phosphines, tricyclohexylphosphine, triethylphosphine, tri-tert-butylphosphine, bis(diphenylphosphino)ethane, bis(diphenylphosphino)propane, bis(diphenylphosphino)butane, bis(diphenylphosphino)heptane, 1,1′-bis(diphenylphosphino)ferrocene, (9,9-dimethyl-9H-xanthene-4,5-diyl)bis(diphenylphosphine), 4,6-bis(diphenylphosphinyl)-10H-phenoxazine, and (oxybis(2,1-phenyl))bis(diphenylphosphane).

11. The process as claimed in claim 10, wherein the metal/ligand ratio is in the range from 1:1 to 1:50 mol/mol.

12. The process as claimed in claim 1, wherein the farnesene:amine ratio used is 1:1 to 1:10 mol/mol.

13. The process as claimed in claim 1, wherein the catalyst and the farnesene are used in a ratio of 1:10 to 1:1000.

14. The process as claimed in claim 1, wherein the reaction is carried out in the presence of an inert gas.

15. The process as claimed in claim 1, wherein the reaction is carried out under pressure in a range from 1 to 10 bar.

16. The process as claimed in claim 1, wherein the reacting of the β-farnesene is carried out at a temperature in the range from 50 to 150° C.

17. The process as claimed claim 1, wherein the reaction time is 1 to 16 hours.

18. The process as claimed in claim 1, wherein the catalyst used is recycled after the reaction.

19. The process as claimed in claim 1, wherein a dimethylammonium carbamate is used as dimethylamine source and a TPPMS, TPPTS or DPPBTS is used as ligand.
